(19) United States
(12) Patent Application Publication
(10) Pub. No.: US 2008/0090273 A1
(43) Pub. Date: Apr. 17, 2008

(54) Malic Acid Production in Recombinant Yeast

(76) Inventors: Aaron Adriaan Winkler, The Hague (NL); Abraham Frederik de Hulster, Pijnacker (NL); Johannes Pieter van Dijken, Leidschendam (NL); Jacobus Thomas Pronk, Schipluiden (NL)

Correspondence Address: WILLIAMS, MORGAN & AMERSON, 10333 RICHMOND, SUITE 1100, HOUSTON, TX, 77042, US

(21) Appl. No.: 11/554,730

(22) Filed: Oct. 31, 2006

Related U.S. Application Data
(60) Provisional application No. 60/738,473, filed on Nov. 21, 2005.

(51) Int. Cl. C12N 1/00; C12P 7/46

(52) U.S. Cl. 435/145; 435/255.2

(57) ABSTRACT

We disclose a recombinant yeast, wherein the yeast is pyruvate decarboxylase enzyme (PDC) activity negative (PDC-negative) and is functionally transformed with a coding region encoding a pyruvate carboxylase enzyme (PYC) wherein the PYC is active in the cytosol, a coding region encoding a malate dehydrogenase enzyme (MDH) wherein the MDH is active in the cytosol and is not inactivated in the presence of glucose, and a coding region encoding a malic acid transporter protein (MAE). We also disclose a method of producing malic acid by culturing such a yeast in a medium comprising a carbon source and a carbon dioxide source and isolating malic acid from the medium.

This application claims priority from U.S. provisional patent application Ser. No. 60/738,473, filed on Nov. 21, 2005, which is incorporated herein by reference.

### BACKGROUND OF THE INVENTION

The present invention relates generally to the industrial use of microorganisms. More particularly, it concerns the production of malic acid or succinic acid by yeast.

The use of microorganisms, such as yeast, in performing industrial processes has taken place serendipitiously for thousands of years and has been a subject of technical inquiry for decades. Yeasts such as S. cerevisiae have been used to produce many different small molecules, including organic acids.

However, one organic acid that has been difficult to produce from yeast, particularly S. cerevisiae, is malic acid. Malic acid, C₄H₆O₅, is a dicarboxylic organic acid that imparts a tart taste to many sour or tart foods, such as green apples and wine. Malic acid is useful to the food processing industry as a source of tartness for use in various foods. At this time, we are not aware of high yield production of malic acid by yeast.

### SUMMARY OF THE INVENTION

In one embodiment, the present invention relates to a recombinant yeast, wherein the yeast is pyruvate decarboxylase enzyme (PDC) activity negative (PDC-negative) and is functionally transformed with a coding region encoding either a pyruvate carboxylase enzyme (PYC) wherein the PYC is active in the cytosol or a phosphoenolpyruvate (PEP) carboxylase wherein the PEP carboxylase is insensitive to inhibition by malate, aspartate, and oxaloacetate, a coding region encoding a malate dehydrogenase enzyme (MDH) wherein the MDH is active in the cytosol and is not inactivated in the presence of glucose, and a coding region encoding a malic acid transporter protein (MAE).

In another embodiment, the present invention relates to a method of producing malic acid or succinic acid including culturing a recombinant yeast, wherein the yeast is pyruvate decarboxylase enzyme (PDC) activity negative (PDC-negative) and is functionally transformed with a coding region encoding either a pyruvate carboxylase enzyme (PYC) wherein the PYC is active in the cytosol or a phosphoenolpyruvate (PEP) carboxylase wherein the PEP carboxylase is insensitive to inhibition by malate, aspartate, and oxaloacetate, a coding region encoding a malate dehydrogenase enzyme (MDH) wherein the MDH is active in the cytosol and is not inactivated in the presence of glucose, and a coding region encoding a malic acid transporter protein (MAE), in a medium comprising a carbon source and a carbon dioxide source; and isolating malic acid or succinic acid from the medium.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows glucose and pyruvate concentrations as a function of culture time as described in Example 1.

FIG. 2 shows malate, glycerol, and succinate concentrations as a function of culture time as described in Example 1.

FIG. 3 is a map of plasmid p426GPDMDH3, as described in Example 1.

FIG. 4 is a map of plasmid pRS2, as described in Example 1.

FIG. 5 is a map of plasmid pRS2ΔMDH3, as described in Example 1.

FIG. 6 is a map of plasmid YEplac112 SpMAE1, as described in Example 1.

FIG. 7 shows the start biomass, the consumption of glucose, and the production of pyruvate in Batch A, Example 2.

FIG. 8 shows the production of malate, glycerol, and succinate in Batch A, Example 2.

FIG. 9 shows the start biomass, the consumption of glucose, and the production of pyruvate in Batch B, Example 2.

FIG. 10 shows the production of malate, glycerol, and succinate in Batch B, Example 2.

FIG. 11 shows the start biomass, the consumption of glucose, and the production of pyruvate in Batch C, Example 2.

FIG. 12 shows the production of malate, glycerol, and succinate in Batch C, Example 2.

### DESCRIPTION OF ILLUSTRATIVE EMBODIMENTS

In one embodiment, the present invention relates to a recombinant yeast, wherein the yeast is pyruvate decarboxylase enzyme (PDC) activity negative (PDC-negative) and is functionally transformed with a coding region encoding either a pyruvate carboxylase enzyme (PYC) wherein the PYC is active in the cytosol or a phosphoenolpyruvate (PEP) carboxylase wherein the PEP carboxylase is insensitive to inhibition by malate, aspartate, and oxaloacetate, a coding region encoding a malate dehydrogenase enzyme (MDH) wherein the MDH is active in the cytosol and is not inactivated in the presence of glucose, and a coding region encoding a malic acid transporter protein (MAE).

Any yeast known in the art for use in industrial processes can be used in the method as a matter of routine experimentation by the skilled artisan having the benefit of the present disclosure. The yeast to be transformed can be selected from any known genus and species of yeast. Yeasts are described by N. J. W. Kreger-van Rij, “The Yeasts,” Vol. 1 of Biology of Yeasts, Ch. 2, A. H. Rose and J. S. Harrison, Eds. Academic Press, London, 1987. In one embodiment, the yeast genus can be Saccharomyces, Zygosaccharomyces, Candida, Hansenula, Kluyveromyces, Debaromyces, Nadsonia, Lipomyces, Torulopsis, Kloeckera, Pichia, Schizosaccharomyces, Trigonopsis, Brettanomyces, Cryptococcus, Trichosporon, Aureobasidium, Lipomyces, Phaffia, Rhodotorula, Yarrowia, or Schwanniomyces, among others. In a further embodiment, the yeast can be a Saccharomyces, Zygosaccharomyces, Kluyveromyces or Pichia spp. In yet a further embodiment, the yeasts can be Saccharomyces cerevisiae. Saccharomyces cerevisiae is a commonly used yeast in industrial processes, but the invention is not limited thereto.

A “recombinant” yeast is a yeast that contains a nucleic acid sequence not naturally occurring in the yeast or an additional copy or copies of an endogenous nucleic acid sequence, wherein the nucleic acid sequence is introduced into the yeast or an ancestor cell thereof by human action. Recombinant DNA techniques are well-known, such as in Sambrook et al., Molecular Genetics: A Laboratory Manual, Cold Spring Harbor Laboratory Press, which provides further information regarding various techniques known in the art and discussed herein. In this embodiment, a coding region of the homologous and/or heterologous gene is isolated from an organism, which possesses the gene. The organism can be a bacterium, a prokaryote, a eukaryote, a microorganism, a fungus, a plant, or an animal.

Genetic material comprising the coding region can be extracted from cells of the organism by any known technique. Thereafter, the coding region can be isolated by any appropriate technique. In one known technique, the coding region is isolated by, first, preparing a genomic DNA library or a cDNA library, and second, identifying the coding region in the genomic DNA library or cDNA library, such as by probing the library with a labeled nucleotide probe selected to be or presumed to be at least partially homologous with the coding region, determining whether expression of the coding region imparts a detectable phenotype to a library microorganism comprising the coding region, or amplifying the desired sequence by PCR. Other known techniques for isolating the coding region can also be used.

“PDC-negative” is used herein to describe a yeast which has a pyruvate decarboxylase activity of less than 0.005 micromol/min mg protein⁻¹ when using the methods previously described by van Maris, A J. A., M. Ah. Luttik, A. A. Winkler, J. P. van Dijken, and J. T. Pronk. 2003. Such a yeast may be referred to as having “no PDC activity.” Overproduction of Threonine Aldolase Circumvents the Biosynthetic Role of Pyruvate Decarboxylase in Glucose-grown Saccharomyces cerevisiae. Appl. Environ. Microbiol. 69:2094-2099. Such a yeast may be referred to herein as having “no PDC activity.”

A yeast which is PDC-negative can be isolated or engineered by any appropriate technique. A large starting population of genetically-diverse yeast may contain natural mutants which are PDC-negative. A starting population can be subjected to mutagenesis or chemostat-based selection. A typical PDC-positive yeast strain comprises (A) at least one PDC structural gene that is capable of being expressed in the yeast strain; (B) at least one PDC regulatory gene that is capable of being expressed in the yeast strain; (C) a promoter of the PDC structural gene; and (D) a promoter of the PDC regulatory gene. In a PDC-negative yeast, one or more of (A)-(D) can be (i) mutated, (ii) disrupted, or (iii) deleted. Mutation, disruption or deletion of one or more of (A)-(D) can, in certain embodiments, contribute to a lack of pyruvate decarboxylase activity.

In one embodiment, the PDC-negative yeast is S. cerevisiae strain TAM (“MATa pdcl(−6,−2)::loxP pdc5(−6,−2)::loxP pdc6(−6,−2)::loxP ura3-52” ura-yeast having no detectable pyruvate decarboxylase activity, C₂ carbon source independent, glucose tolerant).

The pyruvate carboxylase (PYC) can be any enzyme capable of catalyzing the conversion of pyruvate to oxaloacetate (EC 6.4.1.1) wherein the PYC is active in the cytosol. An enzyme need not be identified in the literature as a pyruvate carboxylase at the time of filing of the present application to be within the definition of a PYC. A PYC from any source organism may be used and the PYC may be wild type or modified from wild type. The PYC can be S. cerevisiae pyruvate carboxylase. In one embodiment, the PYC has at least 75% identity to the amino acid sequence given in SEQ ID NO:1.In one embodiment, the PYC has at least 80% identity to the amino acid sequence given in SEQ ID NO:1. In one embodiment, the PYC has at least 85% identity to the amino acid sequence given in SEQ ID NO:1. In one embodiment, the PYC has at least 90% identity to the amino acid sequence given in SEQ ID NO:1. In one embodiment, the PYC has at least 95% identity to the amino acid sequence given in SEQ ID NO:1. In another embodiment, the PYC has at least 96% identity to the amino acid sequence given in SEQ ID NO: 1. In an additional embodiment, the PYC has at least 97% identity to the amino acid sequence given in SEQ ID NO: 1. In yet another embodiment, the PYC has at least 98% identity to the amino acid sequence given in SEQ ID NO: 1. In still another embodiment, the PYC has at least 99% identity to the amino acid sequence given in SEQ ID NO: 1. In still yet another embodiment, the PYC has the amino acid sequence given in SEQ ID NO: 1.

Identity can be determined by a sequence alignment performed using the ClustalW program and its default values, namely: DNA Gap Open Penalty=15.0, DNA Gap Extension Penalty=6.66, DNA Matrix=Identity, Protein Gap Open Penalty=10.0, Protein Gap Extension Penalty=0.2, Protein matrix=Gonnet. Identity can be calculated according to the procedure described by the ClustalW documentation: “A pairwise score is calculated for every pair of sequences that are to be aligned. These scores are presented in a table in the results. Pairwise scores are calculated as the number of identities in the best alignment divided by the number of residues compared (gap positions are excluded). Both of these scores are initially calculated as percent identity scores and are converted to distances by dividing by 100 and subtracting from 1.0 to give number of differences per site. We do not correct for multiple substitutions in these initial distances. As the pairwise score is calculated independently of the matrix and gaps chosen, it will always be the same value for a particular pair of sequences.”

It should be noted that a coding region is considered to be of or from an organism if it encodes a protein sequence substantially identical to that of the same protein purified from cells of the organism.

In one embodiment, the yeast can be transformed with a coding region encoding a phosphoenolpyruvate (PEP) carboxylase, either as an alternative to or in addition to the PYC (EC 4.1.1.38). The PEP carboxylase can be any enzyme capable of catalyzing the conversion of phosphoenolpyruvate to oxaloacetate. An enzyme need not be identified in the literature as a PEP carboxylase at the time of filing of the present application to be within the definition of a PEP carboxylase. A PEP carboxylase from any source organism may be used and the PEP carboxylase may be wild type or modified from wild type. The PEP carboxylase should be insensitive to inhibition by malate, aspartate, and oxaloacetate. E. coli PEP carboxylase has been observed to be inhibited by malate.

The malate dehydrogenase enzyme (MDH) can be any enzyme capable of catalyzing the conversion of oxaloacetate to malate (EC 1.1.1.37), wherein the MDH is active in the cytosol and is not inactivated in the presence of glucose. (The terms “malate” and “malic acid” may be used interchangeably herein except in contexts where one particular ionic species is indicated). An enzyme need not be identified in the literature as a malate dehydrogenase at the time of filing of the present application to be within the definition of an MDH. “Active in the cytosol” means a catalytically-active form of the enzyme is present in the cytosol. “Not inactivated in the presence of glucose” means that catalytic activity of the enzyme is not reduced when exposed to glucose relative to when glucose is absent. An MDH from any source organism may be used and the MDH may be wild type or modified from wild type. In one embodiment, the MDH can be S. cerevisiae MDH1 or S. cerevisiae MDH3. Wild type S. cerevisiae MDH2 is active in the cytosol but is inactivated in the presence of glucose. In one embodiment, the MDH can be a modified S. cerevisiae MDH2 modified (by genetic engineering, posttranslational modification, or any other technique known in the art) to be active in the cytosol and not inactivated in the presence of glucose. In one embodiment, the MDH contains a signaling sequence or sequences capable of targeting the MDH to the cytosol of the yeast or the MDH lacks a signaling sequence or sequences capable of targeting the MDH to an intracellular region of the yeast other than the cytosol. In one embodiment, the MDH can be S. cerevisiae MDH3ΔSKL, in which the coding region encoding the MDH has been altered to delete the carboxy-terminal SKL residues of wild type S. cerevisiae MDH3, which normally target the MDH3 to the peroxisome. In one embodiment, the MDH has at least 75% identity to the amino acid sequence given in SEQ ID NO:2. In one embodiment, the MDH has at least 80% identity to the amino acid sequence given in SEQ ID NO:2. In one embodiment, the MDH has at least 85% identity to the amino acid sequence given in SEQ ID NO:2. In one embodiment, the MDH has at least 90% identity to the amino acid sequence given in SEQ ID NO:2. In one embodiment, the MDH has at least 95% identity to the amino acid sequence given in SEQ ID NO:2. In another embodiment, the MDH has at least 96% identity to the amino acid sequence given in SEQ ID NO: 2. In an additional embodiment, the MDH has at least 97% identity to the amino acid sequence given in SEQ ID NO: 2. In yet another embodiment, the MDH has at least 98% identity to the amino acid sequence given in SEQ ID NO: 2. In still another embodiment, the MDH has at least 99% identity to the amino acid sequence given in SEQ ID NO: 2. In still yet another embodiment, the MDH has the amino acid sequence given in SEQ ID NO: 2.

The malic acid transporter protein (MAE) can be any protein capable of transporting malate from the cytosol of a yeast across the cell membrane and into extracellular space. A protein need not be identified in the literature as a malic acid transporter protein at the time of filing of the present application to be within the definition of an MAE. An MAE from any source organism may be used and the MAE may be wild type or modified from wild type. The MAE can be Schizosaccharomyces pombe SpMAE1. In one embodiment, the MAE has at least 75% identity to the amino acid sequence given in SEQ ID NO:3. In one embodiment, the MAE has at least 80% identity to the amino acid sequence given in SEQ ID NO:3. In one embodiment, the MAE has at least 85% identity to the amino acid sequence given in SEQ ID NO:3. In one embodiment, the MAE has at least 90% identity to the amino acid sequence given in SEQ ID NO:3. In one embodiment, the MAE has at least 95% identity to the amino acid sequence given in SEQ ID NO:3. In another embodiment, the MAE has at least 96% identity to the amino acid sequence given in SEQ ID NO: 3. In an additional embodiment, the MAE has at least 97% identity to the amino acid sequence given in SEQ ID NO: 3. In yet another embodiment, the MAE has at least 98% identity to the amino acid sequence given in SEQ ID NO: 3. In still another embodiment, the MAE has at least 99% identity to the amino acid sequence given in SEQ ID NO: 3. In still yet another embodiment, the MAE has the amino acid sequence given in SEQ ID NO: 3.

Preferably, a coding region encoding a desired enzyme is incorporated into the yeast in such a manner that the desired enzyme is produced in the yeast and is substantially functional. Such a yeast may be referred to herein as being “functionally transformed.”

Once the coding region encoding the enzyme or protein has been extracted from an organism's nucleic acids or synthesized by chemical means, it can be prepared for transformation into and expression in the yeast. At minimum, this involves the insertion of the coding region into a vector and operable linkage to a promoter found on the vector and active in the yeast. Any vector (integrative, chromosomal or episomal) can be used.

Any promoter active in the target host (homologous or heterologous; constitutive, inducible or repressible) can be used. Such insertion can involve the use of restriction endonucleases to “open up” the vector at a desired point where operable linkage to the promoter is possible, followed by ligation of the coding region into the desired point. If desired, before insertion into the vector, the coding region can be prepared for use in the target organism. This can involve altering the codons used in the coding region to more fully match the codon use of the target organism; changing sequences in the coding region that could impair the transcription or translation of the coding region or the stability of an mRNA transcript of the coding region; or adding or removing portions encoding signaling peptides (regions of the protein encoded by the coding region that direct the protein to specific locations (e.g. an organelle, the membrane of the cell or an organelle, or extracellular secretion)), among other possible preparations known in the art.

Regardless whether the coding region is modified, when the coding region is inserted into the vector, it is operably linked to a promoter active in the yeast. A promoter, as is known, is a DNA sequence that can direct the transcription of a nearby coding region. As already described, the promoter can be constitutive, inducible or repressible. Constitutive promoters continually direct the transcription of a nearby coding region. Inducible promoters can be induced by the addition to the medium of an appropriate inducer molecule, which will be determined by the identity of the promoter. Repressible promoters can be repressed by the addition to the medium of an appropriate repressor molecule, which will be determined by the identity of the promoter. In one embodiment, the promoter is constitutive. For example, in a further embodiment, the constitutive promoter is the S. cerevisiae triosephosphateisomerase (TPI) promoter. For another example, in another further embodiment, the promoter can be S. cerevisiae glyceraldehyde-3-phosphate dehydrogenase (isozyme 3) THD3 promoter.

A terminator region can be used, if desired. An exemplary terminator region is S. cerevisiae CYC1.

The vector comprising the coding region operably linked to the promoter can be a plasmid, a cosmid, or a yeast artificial chromosome, among others known in the art to be appropriate for use in yeast. In addition to the coding region operably linked to the promoter, the vector can also comprise other genetic elements. For example, if the vector is not expected to integrate into the yeast genome, the vector can comprise an origin of replication, which allows the vector to be passed on to progeny cells of a yeast comprising the vector. If integration of the vector into the yeast genome is desired, the vector can comprise sequences homologous to sequences found in the yeast genome, and can also comprise coding regions that can facilitate integration. To determine which yeast cells are transformed, the vector can comprise a selectable marker or screenable marker which imparts a phenotype to the yeast that distinguishes it from untransformed yeast, e.g. it survives on a medium comprising an antibiotic fatal to untransformed yeast or it metabolizes a component of the medium into a product that the untransformed yeast does not, among other phenotypes. In addition, the vector may comprise other genetic elements, such as restriction endonuclease sites and others typically found in vectors.

After the vector is prepared, with the coding region operably linked to the promoter, the yeast can be transformed with the vector (i.e. the vector can be introduced into at least one of the cells of a yeast population). Techniques for yeast transformation are well established, and include electroporation, microprojectile bombardment, and the LiAc/ssDNA/PEG method, among others. Yeast cells, which are transformed, can then be detected by the use of a screenable or selectable marker on the vector. It should be noted that the phrase “transformed yeast” has essentially the same meaning as “recombinant yeast,” as defined above. The transformed yeast can be one that received the vector in a transformation technique, or can be a progeny of such a yeast.

Concerning the PYC, MDH, and MAE, the skilled artisan having the benefit of the present disclosure will understand, in light of the redundancy of the genetic code, that a large number of potential coding regions can exist which will encode a particular PYC sequence, MDH sequence, or MAE sequence. An exemplary PYC coding region is given as SEQ ID NO:4; an exemplary MDH coding region is given as SEQ ID NO:5; and an exemplary MAE coding region is given as SEQ ID NO:6. Any coding region which will encode a desired protein sequence may be used as a matter of routine experimentation. The skilled artisan will understand that particular codons (“biased codons”) may have larger corresponding tRNA pools in the yeast than different redundant codons and thus may allow more rapid protein translation in the yeast.

The skilled artisan will also understand that various regulatory sequences, such as promoters and enhancers, among others known in the art, can be used as a matter of routine experimentation in preparation and use of the functionally transformed yeast.

The present invention is not limited to the enzymes of the pathways known for the production of malic acid intermediates or malic acid in plants, yeast, or other organisms.

In another embodiment, the present invention relates to a method of producing malic acid or succinic acid comprising culturing a recombinant yeast, wherein the yeast is pyruvate decarboxylase enzyme (PDC) activity negative (PDC-negative) and is functionally transformed with a coding region encoding a pyruvate carboxylase enzyme (PYC) wherein the PYC is active in the cytosol, a coding region encoding a malate dehydrogenase enzyme (MDH) wherein the MDH is active in the cytosol and is not inactivated in the presence of glucose, and a coding region encoding a malic acid transporter protein (MAE), in a medium comprising a carbon source and a carbon dioxide source; and isolating malic acid or succinic acid from the medium.

The yeast and the coding regions thereof can be as described above.

After a recombinant yeast has been obtained, the yeast can be cultured in a medium. The medium in which the yeast can be cultured can be any medium known in the art to be suitable for this purpose. Culturing techniques and media are well known in the art. In one embodiment, culturing can be performed by aqueous fermentation in an appropriate vessel. Examples for a typical vessel for yeast fermentation comprise a shake flask or a bioreactor.

The medium can comprise a carbon source such as glucose, sucrose, fructose, lactose, galactose, or hydrolysates of vegetable matter, among others. In one embodiment, the medium can also comprise a nitrogen source as either an organic or an inorganic molecule. In a further embodiment, the medium can also comprise components such as amino acids; purines; pyrimidines; corn steep liquor; yeast extract; protein hydrolysates; water-soluble vitamins, such as B complex vitamins; or inorganic salts such as chlorides, hydrochlorides, phosphates, or sulfates of Ca, Mg, Na, K, Fe, Ni, Co, Cu, Mn, Mo, or Zn, among others. Further components known to one of ordinary skill in the art to be useful in yeast culturing or fermentation can also be included. The medium can be buffered but need not be.

The carbon dioxide source can be gaseous carbon dioxide (which can be introduced to a headspace over the medium or sparged through the medium) or a carbonate salt (for example, calcium carbonate).

During the course of the fermentation, the carbon source is internalized by the yeast and converted, through a number of steps, into malic acid. Expression of the MAE allows the malic acid so produced to be secreted by the yeast into the medium. Typically, some amount of the carbon source is converted into succinic acid and some amount of the succinic acid is secreted by the yeast into the medium.

An exemplary medium is mineral medium containing 50 g/L CaCO₃ and 1 g/L urea.

After culturing has progressed for a sufficient length of time to produce a desired concentration of malic acid or succinic acid in the medium, the malic acid or succinic acid can be isolated. “Isolated,” as used herein to refer to an organic acid, means being brought to a state of greater purity by separation of the organic acid from at least one other component (either another organic acid or a compound not in that category) of the yeast or the medium. In one embodiment, the isolated organic acid is at least about 95% pure, such as at least about 99% pure.

To isolate malic acid accumulated in the medium, the isolation can comprise purifying the malic acid from the medium by known techniques, such as the use of an ion exchange resin, activated carbon, microfiltration, ultrafiltration, nanofiltration, liquid-liquid extraction, crystallization, or chromatography, among others.

The isolation of succinic acid can be performed in the same way.

We have observed that culturing a recombinant yeast of the present invention in mineral medium comprising 50 g/L CaCO₃ and 1 g/L urea can lead to levels of malic acid (as acid) in the medium of at least 1 g/L. In one embodiment, it can lead to levels of malic acid (as acid) in the medium of at least 10 g/L. In a further embodiment, it can lead to levels of malic acid (as acid) in the medium of at least 30 g/L.

If the yeast accumulates malic acid in the medium during the culturing step, preferably the concentration of malic acid is stabilized or allowed to increase.

The following definitions are provided in order to aid those skilled in the art in understanding the detailed description of the present invention.

The term “accumulation of malic acid above background levels” refers to the accumulation of malic acid above undetectable levels as determined using the procedures described herein.

“Amplification” refers to increasing the number of copies of a desired nucleic acid molecule or to increase the activity of an enzyme, by whatsoever means.

“Codon” refers to a sequence of three nucleotides that specify a particular amino acid.

“DNA ligase” refers to an enzyme that covalently joins two pieces of double-stranded DNA.

“Electroporation” refers to a method of introducing foreign DNA into cells that uses a brief, high voltage DC charge to permeabilize the host cells, causing them to take up extra-chromosomal DNA.

“Endonuclease” refers to an enzyme that hydrolyzes double stranded DNA at internal locations.

The term “expression” refers to the transcription of a gene to produce the corresponding mRNA and translation of this mRNA to produce the corresponding gene product, i.e., a peptide, polypeptide, or protein.

The phrase “functionally linked” or “operably linked” refers to a promoter or promoter region and a coding or structural sequence in such an orientation and distance that transcription of the coding or structural sequence may be directed by the promoter or promoter region.

The term “gene” refers to chromosomal DNA, plasmid DNA, cDNA, synthetic DNA, or other DNA that encodes a peptide, polypeptide, protein, or RNA molecule, and regions flanking the coding sequence involved in the regulation of expression.

The term “genome” encompasses both the chromosomes and plasmids within a host cell. Encoding DNAs of the present invention introduced into host cells can therefore be either chromosomally integrated or plasmid-localized.

“Heterologous DNA” refers to DNA from a source different than that of the recipient cell.

“Homologous DNA” refers to DNA from the same source as that of the recipient cell.

“Hybridization” refers to the ability of a strand of nucleic acid to join with a complementary strand via base pairing. Hybridization occurs when complementary sequences in the two nucleic acid strands bind to one another.

The term “medium” refers to the chemical environment of the yeast comprising any component required for the growth of the yeast or the recombinant yeast and one or more precursors for the production of ascorbic acid. Components for growth of the yeast and precursors for the production of ascorbic acid may or may be not identical.

“Open reading frame (ORF)” refers to a region of DNA or RNA encoding a peptide, polypeptide, or protein.

“Plasmid” refers to a circular, extra chromosomal, replicatable piece of DNA.

“Polymerase chain reaction (PCR)” refers to an enzymatic technique to create multiple copies of one sequence of nucleic acid. Copies of DNA sequence are prepared by shuttling a DNA polymerase between two amplimers. The basis of this amplification method is multiple cycles of temperature changes to denature, then re-anneal amplimers, followed by extension to synthesize new DNA strands in the region located between the flanking amplimers.

The term “promoter” or “promoter region” refers to a DNA sequence, usually found upstream (5′) to a coding sequence, that controls expression of the coding sequence by controlling production of messenger RNA (mRNA) by providing the recognition site for RNA polymerase and/or other factors necessary for start of transcription at the correct site.

A “recombinant cell” or “transformed cell” is a cell that contains a nucleic acid sequence not naturally occurring in the cell or an additional copy or copies of an endogenous nucleic acid sequence, wherein the nucleic acid sequence is introduced into the cell or an ancestor thereof by human action.

The term “recombinant vector” or “recombinant DNA or RNA construct” refers to any agent such as a plasmid, cosmid, virus, autonomously replicating sequence, phage, or linear or circular single-stranded or double-stranded DNA or RNA nucleotide sequence, derived from any source, capable of genomic integration or autonomous replication, comprising a nucleic acid molecule in which one or more sequences have been linked in a functionally operative manner. Such recombinant constructs or vectors are capable of introducing a 5′ regulatory sequence or promoter region and a DNA sequence for a selected gene product into a cell in such a manner that the DNA sequence is transcribed into a functional mRNA, which may or may not be translated and therefore expressed.

“Restriction enzyme” refers to an enzyme that recognizes a specific sequence of nucleotides in double stranded DNA and cleaves both strands; also called a restriction endonuclease. Cleavage typically occurs within the restriction site or close to it.

“Selectable marker” refers to a nucleic acid sequence whose expression confers a phenotype facilitating identification of cells containing the nucleic acid sequence. Selectable markers include those, which confer resistance to toxic chemicals (e.g. ampicillin, kanamycin) or complement a nutritional deficiency (e.g. uracil, histidine, leucine).

“Screenable marker” refers to a nucleic acid sequence whose expression imparts a visually distinguishing characteristic (e.g. color changes, fluorescence).

“Transcription” refers to the process of producing an RNA copy from a DNA template.

“Transformation” refers to a process of introducing an exogenous nucleic acid sequence (e.g., a vector, plasmid, or recombinant nucleic acid molecule) into a cell in which that exogenous nucleic acid is incorporated into a chromosome or is capable of autonomous replication. A cell that has undergone transformation, or a descendant of such a cell, is “transformed” or “recombinant.” If the exogenous nucleic acid comprises a coding region encoding a desired protein, and the desired protein is produced in the transformed yeast and is substantially functional, such a transformed yeast is “functionally transformed.”

“Translation” refers to the production of protein from messenger RNA.

The term “yield” refers to the amount of malic acid produced (molar or weight/volume) divided by the amount of carbon source consumed (molar or weight/volume) multiplied by 100.

“Unit” of enzyme refers to the enzymatic activity and indicates the amount of micromoles of substrate converted per mg of total cell proteins per minute.

“Vector” refers to a DNA or RNA molecule (such as a plasmid, cosmid, bacteriophage, yeast artificial chromosome, or virus, among others) that carries nucleic acid sequences into a host cell. The vector or a portion of it can be inserted into the genome of the host cell.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

Two yeast strains were constructed starting with S. cerevisiae strain TAM (MATa pdcl(−6,−2)::loxP pdc5(−6,−2)::loxP pdc6(−6,−2)::loxP ura3-52 (PDC-negative)), which was transformed with genes encoding a pyruvate carboxylase (PYC), a malate dehydrogenase (MDH), and a malate transporter protein (MAE).

Because the TAM strain has only one auxotrophic marker, we disrupted the TRP1 locus in order to be able to introduced more than one plasmid with an auxotrophic marker, resulting in RWB961 (MATa pdc1(−6,−2)::loxP pdc5(−6,−2)::loxP pdc6(−6,−2)::loxP mutx ura3-52 trp1::Kanlox).

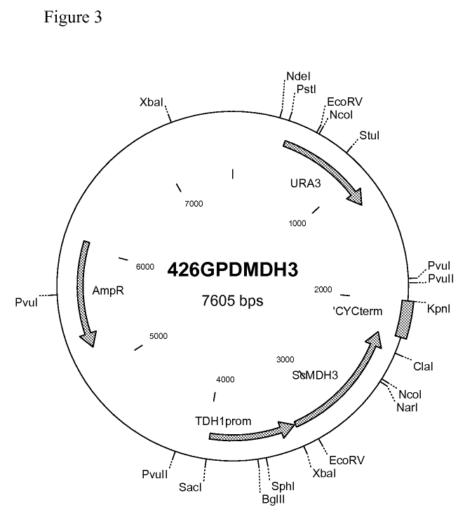

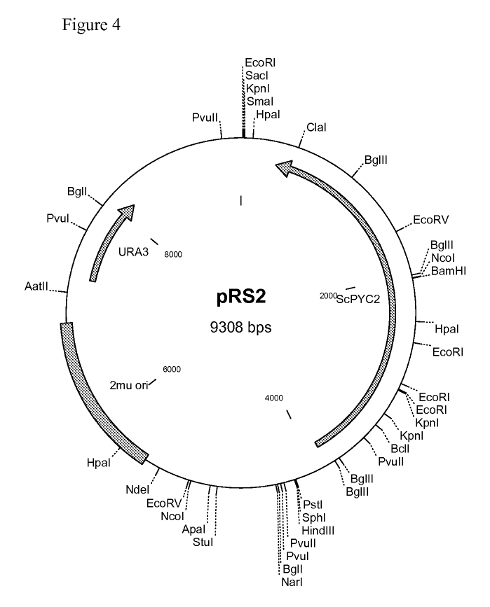

The MDH and PYC genes we used had been previously cloned into plasmids p426GPDMDH3 (2μ plasmid with URA3 marker, containing the MDH3ΔSKL gene between the S. cerevisiae THD3 promoter and the S. cerevisiae CYC1 terminator, FIG. 3) and pRS2 (2μ plasmid with URA3 marker containing the S. cerevisiae PYC2 gene, FIG. 4).

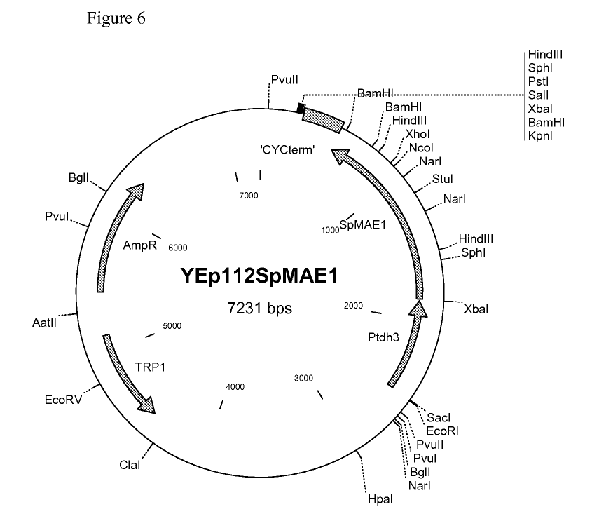

A P_{TDII3}-SpMAE1 cassette carrying the S. pombe MAE was recloned into YEplac112 (2μ, TRP1) and YIplac204 (integration, TRP1), resulting in YEplac112SpMAE1 (FIG. 6) and YIplac204SpMAE1 (not shown).

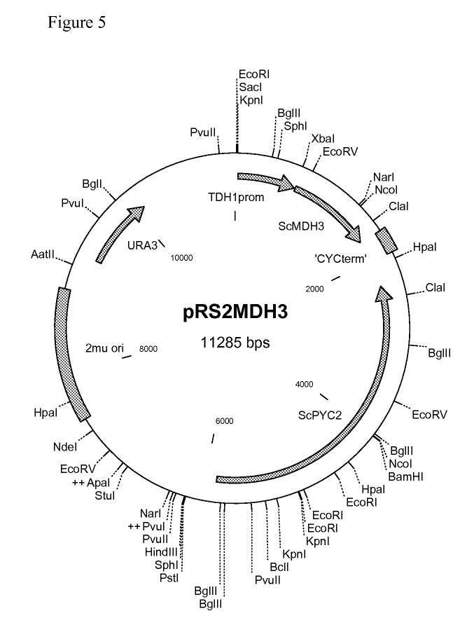

A PYC and MDH vector was prepared: pRS2MDH3ΔSKL (2μ, URA3, PYC2, MDH3ΔSKL) (FIG. 5).

RWB961 was transformed with pRS2MDH3ΔSKL and YEplac112SpMAE1 (strain 1) or pRS2MDH3ΔSKL and YIplac204SpMAE1 (strain 2). Both strain 1 and strain 2 overexpressed PYC2 and MDH3ΔSKL, but had different levels of expression for the MAE1, assuming expression levels were proportional to plasmid copy number, about 10-40 per cell for YEplac112SpMAE1 (2μ-based) and about 1-2 per cell for YIplac204SpMAE1 (integrated).

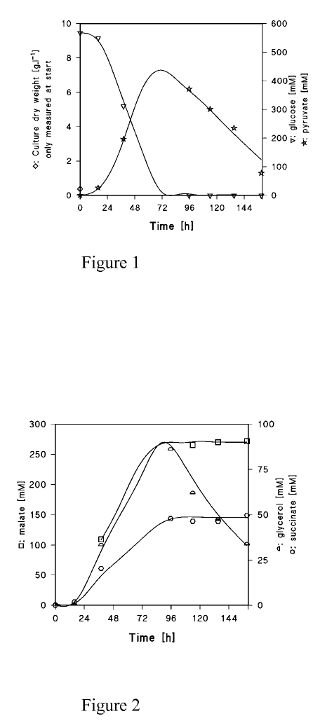

After isolation of strain 1 and strain 2, 0.04 g/L or 0.4 g/L of each strain was introduced to a 500 mL shake flask containing 100 mL mineral medium, 50 g/L CaCO₃, and 1 g/L urea. Flasks were shaken at 200 rpm for the duration of each experiment. Samples of each culture medium were isolated at various times and the concentrations of glucose, pyruvate, glycerol, succinate, and malate determined. Extracellular malate concentrations of about 250 mM after about 90-160 hr were observed. Results are shown in FIGS. 1-2.

The results indicate that the following modifications to yeast metabolic pathways allow high levels of extracellular malate accumulation by recombinant yeasts:

1. Direct the pyruvate flux towards pyruvate carboxylase (by reducing PDC activity)

2. Increase flux through pyruvate carboxylase by overexpressing PYC.

3. Introduce high malate dehydrogenase activities in the cytosol to capture oxaloacetate formed by PYC.

4. Introduce a heterologous malic acid transporter to facilitate export of malate.

FIG. 2 also shows that extracellular succinate concentrations of about 50 mM could be produced simultaneously with the malate production described above.

### EXAMPLE 2

The effect of carbon dioxide on malate production in a fermenter system was studied using a TAM strain overexpressing PYC2, cytosolic MDH3, and a S. pombe MAE1 transporter (YEplac112SpMAE1), as described in Example 1. Three fermenter experiments were performed:

A: Batch cultivations under fully aerobic conditions.

B: Batch cultivations under fully aerobic conditions with a mixture of N₂/O₂/CO₂ of 70%/20%/10%.

C: Batch cultivations under fully aerobic conditions with a mixture of N₂/O₂/CO₂ of 65%/20%/15%.

Protocol

Media

The mineral medium contained 100 g glucose, 3 g KH₂PO₄, 0.5 g MgSO₂.7H₂O and 1 ml trace element solution according to Verduyn et al (Yeast 8: 501-517, 1992) per liter of demineralized water. After heat sterilization of the medium 20 min at 110° C., 1 ml filter sterilized vitamins according to Verduyn et al (Yeast 8: 501-517, 1992) and a solution containing 1 g urea were added per liter. Addition of 0.2 ml per liter antifoam (BDH) was also performed. No CaCO₃ was added.

Fermenter Cultivations

The fermenter cultivations were carried out in bioreactors with a working volume of 1 liter (Applikon Dependable Instruments, Schiedam, The Netherlands). The pH was automatically controlled at pH 5.0 by titration with 2 M potassium hydroxide. The temperature, maintained at 30° C., is measured with a Pt100-sensor and controlled by means of circulating water through a heating finger. The stirrer speed, using two rushton impellers, was kept constant at 800 rpm. For aerobic conditions, an air flow of 0.5 l.min⁻¹ was maintained, using a Brooks 5876 mass-flow controller (Brooks BV, Veenendaal, The Netherlands), to keep the dissolved-oxygen concentration above 60% of air saturation at atmospheric pressure.

In batches B and C, increased carbon dioxide concentration of 10% or 15% while maintaining a good oxygenation was reached by mixing pressurized air 79% N₂+21% O₂ and a gas mixture containing 79% CO₂+21% O₂ (Hoekloos, Schiedam, the Netherlands). The desired percentage of 10% or 15% CO₂, supplied via a Brooks mass-flow controller, was topped up with pressurized air to a fixed total flow rate of 0.5 L/min.

The pH, DOT and KOH/H2SO4 feeds were monitored continuously using an on-line data acquisition & control system (MFCS/Win, Sartorius BBI Systems).

Off-Gas Analysis

The exhaust gas of the fermenter cultivations was cooled in a condenser (2° C.) and dried with a Perma Pure dryer (type PD-625-12P). Oxygen and carbon dioxide concentrations were determined with a Rosemount NGA 2000 gas analyser. The exhaust gas flow rate was measured with a Saga Digital Flow meter (Ion Science, Cambridge). Specific rates of carbon dioxide production and oxygen consumption were calculated as described by van Urk et al (1988, Yeast 8: 501-517).

Sample Preparation

Samples for biomass, substrate and product analysis were collected on ice. Samples of the fermentation broth and cell free samples (prepared by centrifugation at 10,000×g for 10 minutes) were stored at −20° C. for later analysis.

Determinations of Metabolites

HPLC-Determinations

Determination of sugars, organic acids and polyols were determined simultaneously using a Waters HPLC 2690 system equipped with an HPX-87H Aminex ion exclusion column (300×7.8 mm, BioRad) (60° C., 0.6 ml/min 5 mM H2SO4) coupled to a Waters 2487 UV detector and a Waters 2410 refractive index detector.

Enzymatic Metabolite Determinations

In order to verify the HPLC measurements and/or exclude separation errors, L-malic acid was determined with an enzymatic kit (Boehringer-Mannheim, Catalog No. 0 139 068).

Determination of Dry Weight

The dry weight of yeast in the cultures was determined by filtering 5 ml of a culture on a 0.45 μm filter (Gelman Sciences). When necessary, the sample was diluted to a final concentration between 5 and 10 g.l⁻¹. The filters were kept in an 80° C. incubator for at least 24 hours prior to use in order to determine their dry weight before use. The yeast cells in the sample were retained on the filter and washed with 10 ml of demineralized water. The filter with the cells was then dried in a microwave oven (Amana Raderrange, 1500 Watt) for 20 minutes at 50% capacity. The dried filter with the cells was weighed after cooling for 2 minutes. The dry weight was calculated by subtracting the weight of the filter from the weight of the filter with cells.

Determination of Optical Density (OD₆₆₀) The optical density of the yeast cultures was determined at 660 nm with a spectrophotometer; Novaspec II (Amersham Pharmasia Biotech, Buckinghamshire, UK) in 4 ml cuvets. When necessary the samples were diluted to yield an optical density between 0.1 and 0.3.

Batch A: Fully Aerated 21% O₂ (+79% N₂)

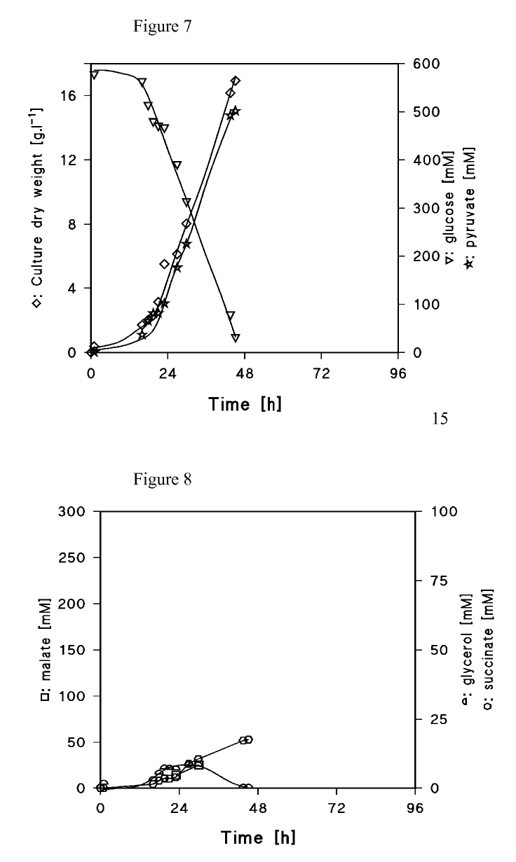

FIGS. 7 and 8 show metabolite formation against time. The result of one representative batch experiment per strain is shown. Replicate experiments yielded essentially the same results. FIG. 7 denotes the start biomass (rectangle), the consumption of glucose (triangle) and the production of pyruvate (star). FIG. 8 denotes production of malate (square), glycerol (upper semi circle), and succinate (octagon). As shown in FIG. 8, the yeast produced about 25 mM malate after 24 hr and about 20 mM succinate after 48 hr.

Batch B: 10% CO₂+21% O₂(+69% N₂)

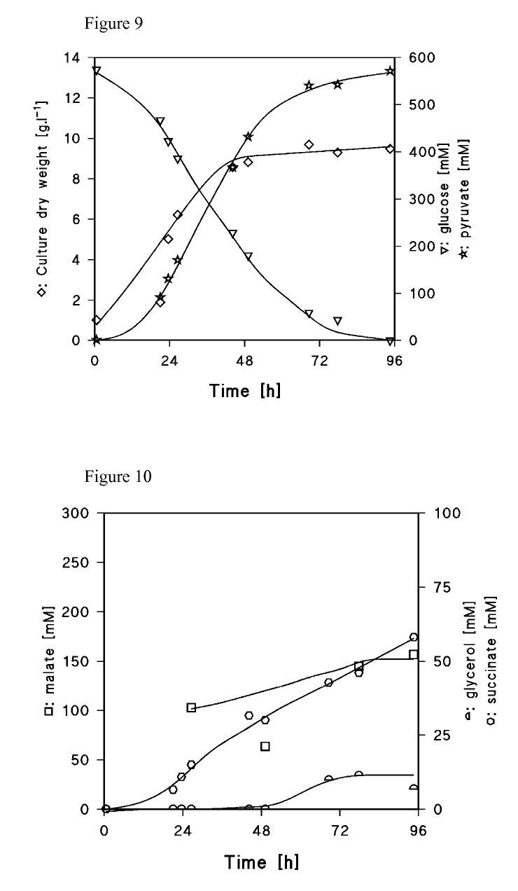

FIGS. 9 and 10 show metabolite formation against time. FIG. 9 denotes the start biomass (rectangle), the consumption of glucose (triangle) and the production of pyruvate (star). FIG. 10 denotes production of malate (square), glycerol (upper semi circle), and succinate (octagon). As shown in FIG. 10, the yeast produced about 100 mM malate after 24 hr and about 150 mM malate after 96 hr, as well as about 60 mM succinate after 96 hr.

Batch C: 15% CO₂+21% O₂(+64% N₂)

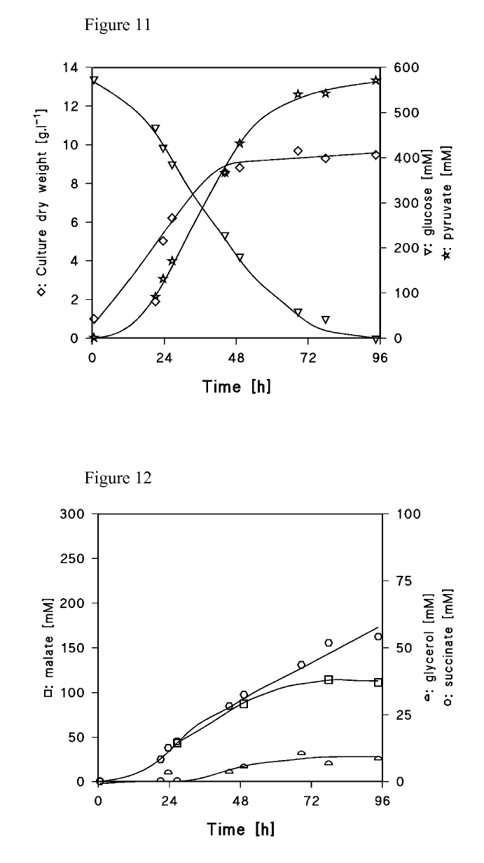

FIGS. 11 and 12 show metabolite formation against time. FIG. 11 denotes the start biomass (rectangle), the consumption of glucose (triangle) and the production of pyruvate (star). FIG. 12 denotes production of malate (square), glycerol (upper semi circle), and succinate (octagon). As shown in FIG. 10, the yeast produced about 45 mM malate after 24 hr and about 100 mM malate after 96 hr, as well as about 60 mM succinate after 96 hr.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A recombinant yeast, wherein the yeast is pyruvate decarboxylase enzyme (PDC) activity negative (PDC-negative) and is functionally transformed with a coding region encoding either a pyruvate carboxylase enzyme (PYC) wherein the PYC is active in the cytosol or a phosphoenolpyruvate (PEP) carboxylase wherein the PEP carboxylase is insensitive to inhibition by malate, aspartate, and oxaloacetate; a coding region encoding a malate dehydrogenase enzyme (MDH) wherein the MDH is active in the cytosol and is not inactivated in the presence of glucose; and a coding region encoding a malic acid transporter protein (MAE).

2. The recombinant yeast of claim 1, wherein the yeast is of the species Saccharomyces cerevisiae.

3. The recombinant yeast of claim 2, wherein the yeast is S. cerevisiae strain TAM.

4. The recombinant yeast of claim 1, wherein the PYC is S. cerevisiae pyruvate carboxylase, the MDH is S. cerevisiae MDH1 or S. cerevisiae MDH3, and the MAE is Schizosaccharomyces pombe SpMAE1.

5. The recombinant yeast of claim 4, wherein the MDH is targeted to the cytosol of the yeast by modification of the coding region encoding the MDH relative to a coding region encoding wild type MDH.

6. The recombinant yeast of claim 1, wherein the PYC has at least 75% identity to SEQ ID NO:1, the MDH has at least 75% identity to SEQ ID NO:2, and the MAE has at least 75% identity to SEQ ID NO:3.

7. The recombinant yeast of claim 6, wherein the PYC has at least 95% identity to SEQ ID NO:1, the MDH has at least 95% identity to SEQ ID NO:2, and the MAE has at least 95% identity to SEQ ID NO:3.

8. The recombinant yeast of claim 6, wherein the PYC has the sequence shown in SEQ ID NO:1, the MDH has the sequence shown in SEQ ID NO:2, and the MAE has the sequence shown in SEQ ID NO:3.

9. A method of producing malic acid, comprising:
culturing a recombinant yeast, wherein the yeast is pyruvate decarboxylase enzyme (PDC) activity negative (PDC-negative) and is functionally transformed with a coding region encoding either a pyruvate carboxylase enzyme (PYC) wherein the PYC is active in the cytosol or a phosphoenolpyruvate (PEP) carboxylase wherein the PEP carboxylase is insensitive to inhibition by malate, aspartate, and oxaloacetate; a coding region encoding a malate dehydrogenase enzyme (MDH) wherein the MDH is active in the cytosol and is not inactivated in the presence of glucose; and a coding region encoding a malic acid transporter protein (MAE), in a medium comprising a carbon source and a carbon dioxide source; and
isolating malic acid from the medium.

10. The method of claim 9, wherein the carbon source is glucose.

11. The method of claim 9, wherein the yeast is of the species Saccharomyces cerevisiae.

12. The method of claim 11, wherein the yeast is S. cerevisiae strain TAM.

13. The method of claim 9, wherein the yeast is functionally transformed with a coding region encoding S. cerevisiae pyruvate carboxylase, a coding region encoding S. cerevisiae MDH1 or S. cerevisiae MDH3, and a coding region encoding Schizosaccharomyces pombe SpMAE1.

14. The method of claim 13, wherein the MDH is targeted to the cytosol of the yeast.

15. The method of claim 9, wherein yeast is functionally transformed with a coding region encoding a PYC having at least 75% identity to SEQ ID NO:1, a coding region encoding an MDH having at least 75% identity to SEQ ID NO:2, and a coding region encoding an MAE having at least 75% identity to SEQ ID NO:3.

16. The method of claim 9, wherein yeast is functionally transformed with a coding region encoding a PYC having at least 95% identity to SEQ ID NO:1, a coding region encoding an MDH having at least 95% identity to SEQ ID NO:2, and a coding region encoding an MAE having at least 95% identity to SEQ ID NO:3.

17. The method of claim 16, wherein the PYC has the sequence shown in SEQ ID NO:1, the MDH has the sequence shown in SEQ ID NO:2, and the MAE has the sequence shown in SEQ ID NO:3.

18. The method of claim 9, further comprising isolating succinic acid from the medium.


SEQUENCE LISTING (6 sequences)

SEQ ID NO: 1 - protein, 1180 aa, Saccharomyces cerevisiae
MSSSKKLAGLRDNFSLLGEKNKILVANRGEIPIRIFRSAHELSMRTIAIYSHEDRLSMHRLKADEAYVIGEEGQYTPVGAYLAMDEIIEIAKKHKVDFIHPGYGFLSENSEFADKVVKAGITWIGPPAEVIDSVGDKVSARHLAARANVPTVPGTPGPIETVQEALDFVNEYGYPVIIKAAFGGGGRGMRVVREGDDVADAFQRATSEARTAFGNGTCFVERFLDKPKHIEVQLLADNHGNVVHLFERDCSVQRRHQKVVEVAPAKTLPREVRDAILTDAVKLAKVCGYRNAGTAEFLVDNQNRHYFIEINPRIQVEHTITEEITGIDIVSAQIQIAAGATLTQLGLLQDKITTRGFSIQCRITTEDPSKNFQPDTGRLEVYRSAGGNGVRLDGGNAYAGATISPHYDSMLVKCSCSGSTYEIVRRKMIRALIEFRIRGVKTNIPFLLTLLTNPVFIEGTYWTTFIDDTPQLFQMVSSQNRAQKLLHYLADLAVNGSSIKGQIGLPKLKSNPSVPHLHDAQGNVINVTKSAPPSGWRQVLLEKGPSEFAKQVRQFNGTLLMDTTWRDAHQSLLATRVRTHDLATIAPTTAHALAGAFALECWGGATFDVAMRFLHEDPWERLRKLRSLVPNIPFQMLLRGANGVAYSSLPDNAIDHFVKQAKDNGVDIFRVFDALNDLEQLKVGVNAVKKAGGVVEATVCYSGDMLQPGKKYNLDYYLEVVEKIVQMGTHILGIKDMAGTMKPAAAKLLIGSLRTRYPDLPIHVHSHDSAGTAVASMTACALAGADVVDVAINSMSGLTSQPSINALLASLEGNIDTGINVEHVRELDAYWAEMRLLYSCFEADLKGPDPEVYQHEIPGGQLTNLLFQAQQLGLGEQWAETKRAYREANYLLGDIVKVTPTSKVVGDLAQFMVSNKLTSDDIRRLANSLDFPDSVMDFFEGLIGQPYGGFPEPLRSDVLRNKRRKLTCRPGLELEPFDLEKIREDLQNRFGDIDECDVASYNMYPRVYEDFQKIRETYGDLSVLPTKNFLAPAEPDEEIEVTIEQGKTLIIKLQAVGDLNKKTGQREVYFELNGELRKIRVADKSQNIQSVAKPKADVHDTHQIGAPMAGVIIEVKVHKGSLVKKGESIAVLSAMKMEMVVSSPADGQVKDVFIKDGESVDASDLLVVLEEETLPPSQKK

SEQ ID NO: 2 - protein, 340 aa, S. cerevisiae
MVKVAILGASGGVGQPLSLLLKLSPYVSELALYDIRAAEGIGKDLSHINTNSSCVGYDKDSIENTLSNAQVVLIPAGVPRKPGLTRDDLFKMNAGIVKSLVTAVGKFAPNARILVISNPVNSLVPIAVETLKKMGKFKPGNVMGVTNLDLVRAETFLVDYLMLKNPKIGQEQDKTTMHRKVTVIGGHSGETIIPIITDKSLVFQLDKQYEHFIHRVQFGGDEIVKAKQGAGSATLSMAFAGAKFAEEVLRSFHNEKPETESLSAFVYLPGLKNGKKAQQLVGDNSIEYFSLPIVLRNGSVVSIDTSVLEKLSPREEQLVNTAVKELRKNIEKGKSFILDS

SEQ ID NO: 3 - protein, 438 aa, Schizosaccharomyces pombe
MGELKEILKQRYHELLDWNVKAPHVPLSQRLKHFTWSWFACTMATGGVGLIIGSFPFRFYGLNTIGKIVYILQIFLFSLFGSCMLFRFIKYPSTIKDSWNHHLEKLFIATCLLSISTFIDMLAIYAYPDTGEWMVWVIRILYYIYVAVSFIYCVMAFFTIFNNHVYTIETASPAWILPIFPPMICGVIAGAVNSTQPAHQLKNMVIFGILFQGLGFWVYLLLFAVNVLRFFTVGLAKPQDRPGMFMFVGPPAFSGLALINIARGAMGSRPYIFVGANSSEYLGFVSTFMAIFIWGLAAWCYCLAMVSFLAGFFTRAPLKFACGWFAFIFPNVGFVNCTIEIGKMIDSKAFQMFGHIIGVILCIQWILLMYLMVRAFLVNDLCYPGKDEDAHPPPKPNTGVLNPTFPPEKAPASLEKVDTHVTSTGGESDPPSSEHESV

SEQ ID NO: 4 - DNA, 4144 bp, S. cerevisiae
tcgagctatttattataagagtcagaattggcgcagggagtgttaagtaagaagtactccccatcggatatttcctattgtgtttctgtgatttttagtccttttttcttttctcttactttcggtatccttacttgattacatacataaacaagcccctcttttcttccaaactcttgtagcttactatctgtggcccgtcatttgagtttgatttttttgccaattactatattgcaaaataaaggacagttactaggagagaaaataagggacatagagaacaaaataaaatatgagcagtagcaagaaattggccggtcttagggacaatttcagtttgctcggcgaaaagaataagatcttggtcgccaatagaggtgaaattccgattagaatttttagatctgctcatgagctgtctatgagaaccatcgccatatactcccatgaggaccgtctttcaatgcacaggttgaaggcggacgaagcgtatgttatcggggaggagggccagtatacacctgtgggtgcttacttggcaatggacgagatcatcgaaattgcaaagaagcataaggtggatttcatccatccaggttatgggttcttgtctgaaaattcggaatttgccgacaaagtagtgaaggccggtatcacttggatcggccctccagctgaagttattgactctgtgggtgacaaagtctctgccagacacttggcagcaagagctaacgttcctaccgttcccggtactccaggacctatcgaaactgtgcaagaggcacttgacttcgttaatgaatacggctacccggtgatcattaaggccgcctttggtggtggtggtagaggtatgagagtcgttagagaaggtgacgacgtggcagatgcctttcaacgtgctacctccgaagcccgtactgccttcggtaatggtacctgctttgtggaaagattcttggacaagccaaagcatattgaagttcaattgttggctgataaccacggaaacgtggttcatcttttcgaaagagactgttctgtgcaaagaagacaccaaaaagttgtcgaagtcgctccagcaaagactttgccccgtgaagttcgtgacgctattttgacagatgctgttaaattagctaaggtatgtggttacagaaacgcaggtaccgccgaattcttggttgacaaccaaaacagacactatttcattgaaattaatccaagaattcaagtggagcataccatcactgaagaaatcaccggtattgacattgtttctgcccaaatccagattgccgcaggtgccactttgactcaactaggtctattacaggataaaatcaccacccgtgggttttccatccaatgtcgtattaccactgaagatccctctaagaatttccaaccggataccggtcgcctggaggtctatcgttctgccggtggtaatggtgtgagattggacggtggtaacgcttatgcaggtgctactatctcgcctcactacgactcaatgctggtcaaatgttcatgctctggttctacttatgaaatcgtccgtaggaagatgattcgtgccctgatcgaattcagaatcagaggtgttaagaccaacattcccttcctattgactcttttgaccaatccagtttttattgagggtacatactggacgacttttattgacgacaccccacaactgttccaaatggtatcgtcacaaaacagagcgcaaaaactgttacactatttggcagacttggcagttaacggttcttctattaagggtcaaattggcttgccaaaactaaaatcaaatccaagtgtcccccatttgcacgatgctcagggcaatgtcatcaacgttacaaagtctgcaccaccatccggatggagacaagtgctactggaaaagggaccatctgaatttgccaagcaagtcagacagttcaatggtactctactgatggacaccacctggagagacgctcatcaatctctacttgcaacaagagtcagaacccacgatttggctacaatcgctccaacaaccgcacatgcccttgcaggtgctttcgctttagaatgttggggtggtgctacattcgacgttgcaatgagattcttgcatgaggatccatgggaacgtctgagaaaattaagatctctggtgcctaatattccattccaaatgttattacgtggtgccaacggtgtggcttactcttcattacctgacaatgctattgaccattttgtcaagcaagccaaggataatggtgttgatatatttagagtttttgatgccttgaatgatttagaacaattaaaagttggtgtgaatgctgtcaagaaggccggtggtgttgtcgaagctactgtttgttactctggtgacatgcttcagccaggtaagaaatacaacttagactactacctagaagttgttgaaaaaatagttcaaatgggtacacatatcttgggtattaaggatatggcaggtactatgaaaccggccgctgccaaattattaattggctccctaagaaccagatatccggatttaccaattcatgttcacagtcatgactccgcaggtactgctgttgcgtctatgactgcatgtgccctagcaggtgctgatgttgtcgatgtagctatcaattcaatgtcgggcttaacttcccaaccatcaattaatgcactgttggcttcattagaaggtaacattgatactgggattaacgttgagcatgttcgtgaattagatgcatactgggccgaaatgagactgttgtattcttgtttcgaggccgacttgaagggaccagatccagaagtttaccaacatgaaatcccaggtggtcaattgactaacttgttattccaagctcaacaactgggtcttggtgaacaatgggctgaaactaaaagagcttacagagaagccaattacctactgggagatattgttaaagttaccccaacttctaaggttgtcggtgatttagctcaattcatggtttctaacaaactgacttccgacgatattagacgtttagctaattctttggactttcctgactctgttatggacttttttgaaggtttaattggtcaaccatacggtgggttcccagaaccattaagatctgatgtattgagaaacaagagaagaaagttgacgtgccgtccaggtttagaattagaaccatttgatctcgaaaaaattagagaagacttgcagaacagattcggtgatattgatgaatgcgatgttgcttcttacaatatgtatccaagggtctatgaagatttccaaaagatcagagaaacatacggtgatttatcagttctaccaaccaaaaatttcctagcaccagcagaacctgatgaagaaatcgaagtcaccatcgaacaaggtaagactttgattatcaaattgcaagctgttggtgacttaaataagaaaactgggcaaagagaagtgtattttgaattgaacggtgaattaagaaagatcagagttgcagacaagtcacaaaacatacaatctgttgctaaaccaaaggctgatgtccacgatactcaccaaatcggtgcaccaatggctggtgttatcatagaagttaaagtacataaagggtctttggtgaaaaagggcgaatcgattgctgttttgagtgccatgaaaatggaaatggttgtctcttcaccagcagatggtcaagttaaagacgttttcattaaggatggtgaaagtgttgacgcatcagatttgttggttgtcctagaagaagaaaccctacccccatcccaaaaaaagtaatttttactcgttaattatattttatgacatctgaaaatactagctgtactatatatggcgtatattttatctagttatgttcccatgtatatttaaatgccaaatagaaagtaatcaaacactttcgatgaaatacgtgctaactgtgtttcttccttaatgctttcacttaccatgtctccattctccattttcttcttgagtgaaaatgtgagtttataacgctcaagtacgttaactactctatttaatatcgtacgggatttttgatcgactgtaggttttcttcttagaccattccagcgc

SEQ ID NO: 5 - DNA, 1044 bp, S. cerevisiae
gctctagaaacatggtcaaagtcgcaattcttggcgcttctggtggcgtgggacaaccgctatcattactgctaaaattaagcccttacgtttccgagctggcgttgtacgatatccgagctgcggaaggcattggtaaggatttatctcacatcaacaccaactcaagttgtgtcggttatgataaggatagtattgagaacaccttgtcaaatgctcaggtggtgctaataccggctggtgttcccagaaagcccggtttaactagagatgatttgttcaagatgaacgccggtattgtcaaaagcctggtaaccgctgttggaaagttcgcaccaaatgcgaggattttagtcatttcaaaccctgtaaacagtttggtccctattgctgtggaaactttgaagaaaatgggtaagttcaaacctggaaacgttatgggtgtgacgaaccttgacctggtacgtgcagaaacctttttggtagattatttgatgctaaaaaaccccaaaattggacaagaacaagacaaaactacaatgcacagaaaggtcactgttattgggggtcattcaggggaaaccattatcccaataatcaccgacaaatcgctggtatttcaacttgataagcagtacgagcacttcattcatagggtccagttcggaggtgatgaaattgtcaaagctaaacagggcgccggttccgccacgttgtccatggcgttcgcgggggccaagtttgctgaagaagttttgaggagcttccataatgagaaaccagaaacggagtcactttccgcattcgtttatttaccaggcttaaaaaacggtaagaaagcgcagcaattagttggcgacaactctattgagtatttttccttgccaattgttttgagaaatggtagcgtagtatccatcgataccagtgttctggaaaaactgtctccgagagaggaacaactcgttaatactgcggtcaaagagctacgcaagaatattgaaaaaggcaagagtttcatcctagactcttgagtcgacagct

SEQ ID NO: 6 - DNA, 1336 bp, Schizosaccharomyces pombe
gctctagacatgggtgaactcaaggaaatcttgaaacagaggtatcatgagttgcttgactggaatgtcaaagcccctcatgtccctctcagtcaacgactgaagcattttacatggtcttggtttgcatgtactatggcaactggtggtgttggtttgattattggttctttcccctttcgattttatggtcttaatacaattggcaaaattgtttatattcttcaaatctttttgttttctctctttggatcatgcatgctttttcgctttattaaatatccttcaactatcaaggattcctggaaccatcatttggaaaagcttttcattgctacttgtcttctttcaatatccacgttcatcgacatgcttgccatatacgcctatcctgataccggcgagtggatggtgtgggtcattcgaatcctttattacatttacgttgcagtatcctttatatactgcgtaatggctttttttacaattttcaacaaccatgtatataccattgaaaccgcatctcctgcttggattcttcctattttccctcctatgatttgtggtgtcattgctggcgccgtcaattctacacaacccgctcatcaattaaaaaatatggttatctttggtatcctctttcaaggacttggtttttgggtttatcttttactgtttgccgtcaatgtcttacggttttttactgtaggcctggcaaaaccccaagatcgacctggtatgtttatgtttgtcggtccaccagctttctcaggtttggccttaattaatattgcgcgtggtgctatgggcagtcgcccttatatttttgttggcgccaactcatccgagtatcttggttttgtttctacctttatggctatttttatttggggtcttgctgcttggtgttactgtctcgccatggttagctttttagcgggctttttcactcgagcccctctcaagtttgcttgtggatggtttgcattcattttccccaacgtgggttttgttaattgtaccattgagataggtaaaatgatagattccaaagctttccaaatgtttggacatatcattggggtcattctttgtattcagtggatcctcctaatgtatttaatggtccgtgcgtttctcgtcaatgatctttgctatcctggcaaagacgaagatgcccatcctccaccaaaaccaaatacaggtgtccttaaccctaccttcccacctgaaaaagcacctgcatctttggaaaaagtcgatacacatgtcacatctactggtggtgaatcggatcctcctagtagtgaacatgaaagcgtttaagtcgacgcgt